(19) United States
(12) Patent Application Publication
(10) Pub. No.: US 2015/0018763 A1
(43) Pub. Date: Jan. 15, 2015

(54) Hemorrhoid Pain Relief and Healing Pack

(71) Applicant: Molly Lee, Bellevue, NE (US)

(72) Inventor: Molly Lee, Bellevue, NE (US)

(21) Appl. No.: 13/942,571

(22) Filed: Jul. 15, 2013

(51) Int. Cl. A61F 5/00; A61L 15/44

(52) U.S. Cl. CPC A61F 5/0093; A61L 15/44; A61F 2005/4402
USPC 604/113

(57) ABSTRACT

A hemorrhoid pain relief and healing pack includes a small oval shaped gel filled cold pack inserted within an ultra-soft disposable cloth sleeve infused with 100% natural witch-hazel astringent and is placed over the hemorrhoid bulge/s for a set time of 20 minutes, offering instant pain relief. The flexibility of the frozen gel cold pack allows the product to mold to the specific size/shape of the hemorrhoid bulge/s creating a comfortable supportive process and the cold therapy combined with the soothing and shrinking properties of the ultra-soft witch-hazel infused sleeve offer instant pain relief as well as promoting healing of the affected area. The pack is easy to use, comfortable, and the combination of ingredients used helps to shrink the hemorrhoid bulge/s as well as soothe current pain, swelling, itching, and irritation. The protective sleeve is disposable and therefore sanitary. The product is simple and effective and the cost to manufacture is inexpensive which will allow suffers access to relief no matter their financial status.

### BACKGROUND OF THE INVENTION

1. Field of Invention

The present invention relates to hemorrhoid pain relief and healing products, and more specifically to hemorrhoid pain relief and healing products that combine cold pack therapy with the soothing properties of witch-hazel. The products reduce the user's pain by cooling and covering the area with a healing astringent that aides in soothing and shrinking hemorrhoids.

2. Description of Related Art

“Hemorrhoids result from dilation of the submucosal vascular tissue in the distal anal canal. This vascular tissue is supported by connective tissue that, when weakened, leads to descent or prolapse of the hemorrhoids. Internal hemorrhoids originate above the dentate line (i.e., the junction between columnar and squamous epithelium) and are viscerally innervated and, therefore, painless. External hemorrhoids originate below the dentate line, have somatic innervation, and can cause pain. Some patients have both internal and external (mixed) hemorrhoids.

Internal hemorrhoids are classified according to their degree of prolapse. Grade 1 hemorrhoids may bleed but do not protrude; grade 2 hemorrhoids protrude with defecation but reduce spontaneously; grade 3 hemorrhoids protrude and must be reduced by hand; and grade 4 hemorrhoids are permanently prolapsed.” (http://www.aafp.org/afp/2011/0715/p204.html) “Fifty percent of Americans have hemorrhoids by age 50, according to the American College of Gastroenterology. This group of people ranges from middle aged to those over fifty and they are also extremely common in pregnant women.” (http://www.thehealthjournals.com/archive.php? id=453)

The use of cold therapy for the treatment and shrinkage of hemorrhoids is known in the field. For example, U.S. Pat. No. 20040126412 discloses a small, cylindrical-shaped, flexible bladder that is cooled or frozen before applied to the anus for instant relief and shrinkage of hemorrhoidal tissue. U.S. Pat. No. 20090076575 discloses a cold pack apparatus for abstracting heat to reduce pain for an extended period of time of one hour or more for joint pain or a diseased area, such as hemorrhoids. Both devices can be uncomfortable because applying a frozen pack directly to the affected area without a soft disposable protective sleeve can cause more pain. Both devices pose numerous hygienic problems when re-used on an area that involves potential high bacteria traffic. Besides the cooling agent, neither device provides any additional pain relieving or healing features.

Other devices are designed to relieve the pressure caused by hemorrhoids by using a small tube filled with cool liquid to separate the butt cheeks from the affected area. For instance U.S. Pat. No. 20130046328 discloses a fluid filled tube that produces gentle support and pressure over the hemorrhoid in order to lower pressure across the wall of the hemorrhoid and provide instant pain relief. While this device has a cool liquid inside, it isn't frozen, and therefore would do little in the way of shrinking hemorrhoids. Other than the tube separating the butt cheeks to reduce friction, this device offers no other forms of pain relief or healing properties.

U.S. Pat. No. 20070078417 discloses hemorrhoid relief through a systematic approach and for comprehensive treatment for hemorrhoid sufferers that includes educational materials, a cream for early stages for relief of minor symptoms, fiber pad wipes, cleanser, and the option of a prescription strength ointment for relief of more severe symptoms in relation to swelling. This method is not only time consuming but the use of topical creams and ointments can be messy as well as produce an undesirable odor, making the available times for use limited.

Therefore, there is still an overwhelming need for a hemorrhoid relief product that is simple, effective, safe, sanitary, and accessible to the population no matter their financial situation.

The hemorrhoid pain relief and healing pack according to the present invention meet all the criteria mentioned in [0008] something the aforementioned patents don't.

### SUMMARY OF THE INVENTION

The primary objective of the present invention is to offer a simple and effective product to relieve the pain caused by hemorrhoids.

Another objective of the present invention is to offer a simple and effective product that heals hemorrhoids.

Another objective of the present invention is to offer a product that is safe and non-invasive in the treatment of hemorrhoids.

Yet another objective of the present invention is to offer a product that is disposable, eliminating hygienic concerns.

Still another objective of the present invention is to offer a product that is easy to manufacture and is low-cost.

Finally, the present invention should provide a product that is safe, effective, and easily assessable to the population, regardless of gender, age, or financial status.

The product of the present invention is made up of the following materials:

1) An oval shaped gel filled cold pack approximately 3×5 inches in size

2) An ultra-soft disposable protective cloth sleeve approximately 3¼ inches in width, 4 ½ inches in length, and consisting of a 1 inch fold for pocket insertion

3) Approximately 10 ml of 100% witch-hazel which the above mentioned cloth is soaked in, allowing to infuse

The cold pack may be in different shapes, though preferably in an oval or round shape measuring around 3×5 inches, and include but not limited to freezable gel or liquid that is flexible, allowing for easy conformity to the hemorrhoidal bulge/s and alleviating pain with cold therapy. The protective disposable sleeve in which the cold pack is inserted is made of but not limited to an ultra-soft disposable cloth measuring around 3¼ inches in width, 4½ inches in length, and consisting of a 1 inch fold for pocket insertion, and is infused with but not restricted to 10 ml of 100% all natural witch-hazel. The soft disposable cloth offers a smooth and comforting buffer between the frozen cold pack and the hemorrhoidal bulge/s, which provides relief from itching, burning, and swelling, and the witch-hazel acts as a soothing astringent that aides in the shrinking of hemorrhoids. The prototype of the product is constructed using one 3×5 gel cold pack and one 3 1/4×4 1/2 x 1 inch ultra-soft disposable sleeve infused with 10 ml of 100% all natural witch-hazel.

The product of the present invention offers pain relief through three mechanisms. First, the flexibility of the cold pack conforms to the specific shape of the user's hemorrhoidal bulge/s, providing a subtle support to the affected area. Secondly, the cooling effect from the frozen gel within the cold pack offers instant pain relief due to swelling, itching, and burning. Thirdly, the ultra-soft disposable sleeve infused with witch-hazel provides not only a comforting buffer between the frozen cold pack and the area, but also offers the soothing properties of the witch-hazel astringent, which relieves irritation from burning and itching as well as aides in the shrinkage of hemorrhoids.

The product of the present invention not only offers pain relief of external hemorrhoids, it aides in the actual healing of hemorrhoids through the combined use of cold therapy and witch-hazel astringent to reduce swelling and prevent further bulge/s from prolapsing.

The product is easy to use. The person suffering from hemorrhoids takes the frozen cold pack out of the freezer, slips it in one of the disposable protective sleeves, and places it directly onto the hemorrhoid/s; this can be done in a sitting position or while laying on one's side. The product works instantly by conforming to the hemorrhoidal bulge/s, cooling the entire area, and coating the hemorrhoids with the infused witch-hazel.

The elements for the product of the present invention are simple and easy to manufacture, making the cost to construct the product low, and allow for accessibility to a wider market of those who suffer from hemorrhoids.

Before further explanation of the embodiments of the product of the present invention are given in detail, it is to be understood that the product isn't limited in it's application to the specifics of construction or that of the details set forth in the descriptions or drawings. The product of the present invention can have other embodiments and of being practiced and carried out in various ways. Also the terminology and phraseology used above and following are for the purpose of description and shouldn't be regarded as restricting or limiting.

These descriptions can be utilized as a basis for the designing of other structures, methods, and systems for carrying out several purposes of the present invention. It is important that these claims be regarded as including equivalent constructions as long as they don't stray far from the scope and spirit of the product of the present invention.

The above has outlined in broad terms the preferred features of the product of the present invention so that a better understanding of the detailed descriptions of the product follows. Subsequent features of the invention will be described below that form the subject of claims of the product of the present invention. The disclosed conception and specific embodiment of the product can be readily used as a basis for designing or modifying other structures carrying out the same purposes of the present invention and these other structures should not stray far from the scope and spirit of the product of the present invention in its broadest form.

### DETAILED DESCRIPTION OF THE DRAWINGS

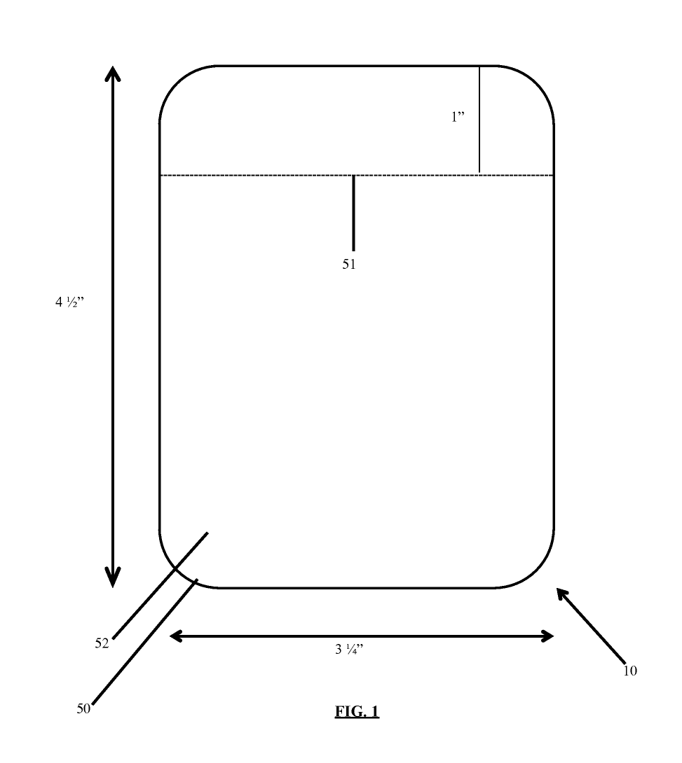

Referring to FIG. 1, there is a birds-eye view of an embodiment of the first portion of the present invention. The protective sleeve of the device 10 is made of a soft disposable cloth 50 with a 1″ pocket opening 51 allowing for insertion of the cold pack. The protective sleeve of the device 10 is a rounded square shape approximately 4½ inches in length, about 3¼ inches in width, and having a 1 inch fold allowing for easy insertion of the cold pack. The protective sleeve of the device 10 is made of a soft disposable cloth 50 that offers a comforting buffer between the cold pack and the hemorrhoid bulge and is infused with approximately 10 ml of 100% natural witch hazel 52 which aides in soothing and shrinking the hemorrhoid bulge. The disposable protective sleeve 10 also covers the entire cold pack keeping the combined product hygienic.

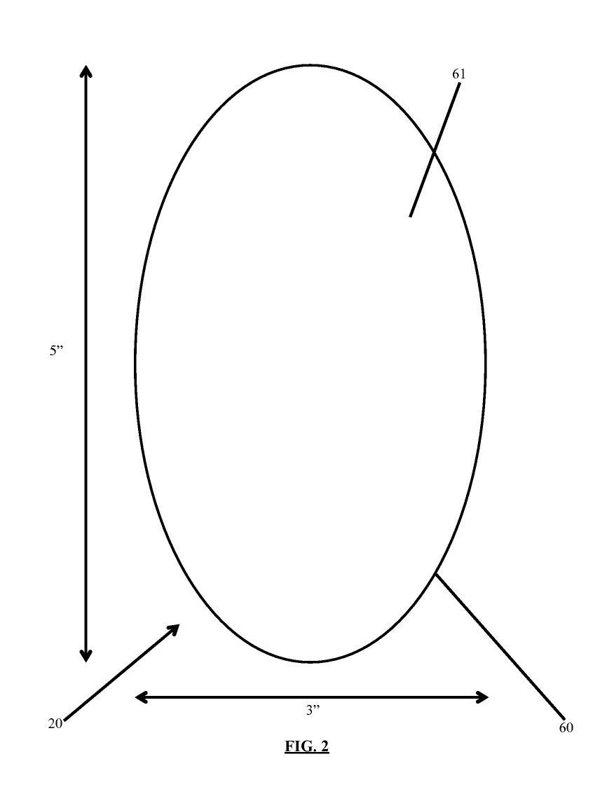

Referring to FIG. 2, there is a birds-eye view of an embodiment of the second portion of the present invention. The cold pack of the device 20 is an oval shape 60 made of strong yet flexible material capable of holding fluid 61 including but not restricted to gel or liquid inside under pressure. The cold pack portion of the device 20 is in an oval shape 60 approximately 5″ in length and 3″ in width and and due to the flexible nature of the cold pack, it easily fits within the dimensions of the protective sleeve 10 which cover the pack completely in order to keep it entirely hygienic. The device 20 contains fluid 61 that is flexible even when frozen, allowing it to conform comfortably over the hemorrhoidal bulge/s, and uses cold therapy to ease the pain and shrink the hemorrhoids.

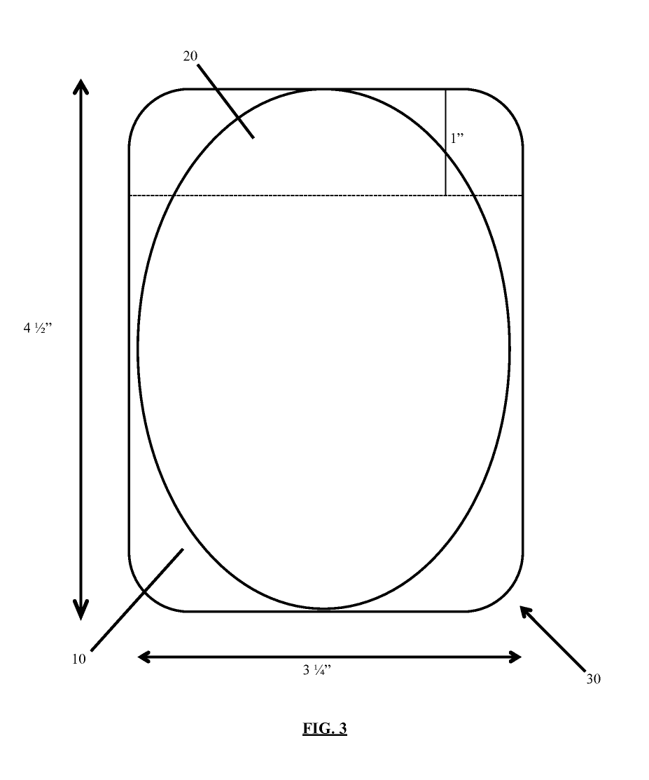

FIG. 3 is a perspective view of the complete embodiments of the device according to the present invention. The cold pack device 20 is an oval shape 60 made of strong yet flexible material capable of holding fluid 61 including but not restricted to gel or liquid inside under pressure and measures approximately 5″ in length and 3″ in width. Due to the flexibility of the cold pack 20 it is easily inserted within the protective sleeve device 10 through the 1″ inch pocket opening 51 which is made of a soft disposable cloth 50 and measures approximately 4½ inches in length, about 3¼ inches in width, and having a 1 inch pocket fold and is infused with approximately 10 ml of 100% natural witch hazel 52. The combined embodiments illustrated in FIG. 3 show all aspects of the present invention which when used as a whole offer pain relief from irritation, itching, and swelling caused by hemorrhoids through the combined use of cold therapy and a soft disposable cloth sleeve infused with 100% witch hazel.

The shown, described, and labeled fundamental features of the present invention as applied to the preferred embodiments, should be taken as and understood that the foregoing is considered purely illustrative only of the principles of the invention and not meant to be taken as limiting or exhausting the invention to the precise or exact forms disclosed. Obvious modifications and variations are possible in light of the above teachings. These embodiments described and detailed above were used to provide the best illustration of the principles of the invention and its practical application to allow one of ordinary skill in the art to utilize the present invention in a variety of embodiments and modifications as are suited to the specific use discussed. Any such modifications or variances are within the scope of the invention as determined by the above claims when interpreted in accordance with the breadth to which they are entitled.

1. A hemorrhoid pain relief and healing pack comprises: a 3×5 inch gel filled oval cold pack; the cold pack made of smooth yet strong material capable of containing freezable gel or liquid under pressure; an 3¼×4½×1 ultra-soft disposable cloth sleeve infused with 10 ml of 100% natural witch-hazel. The product, after being frozen and placed within the disposable sleeve, is placed over the hemorrhoid bulge/s and conforms to their shape, provides instant cooling relief of pain due to swelling, itching, and burning, while the witch-hazel acts as a soothing astringent, aiding in the shrinking and healing of present hemorrhoids.

2. The hemorrhoid pain relief and healing pack of claim 1, wherein the small gel filled cold pack being oval in shape approximately 5 inches in length and 3 inches wide.

3. The hemorrhoid pain relief and healing pack of claim 1, wherein the cold pack may be in a variety of shapes and sizes.

4. The hemorrhoid pain relief and healing pack of claim 1, wherein the material of the sleeve is disposable cloth.

5. The hemorrhoid pain relief and healing pack of claim 1, wherein the frozen cold pack and the disposable witch-hazel infused sleeve are used together acts as an instant pain reliever as well as a product used for healing and shrinking current hemorrhoids.

6. The hemorrhoid pain relief and healing pack of claim 1, wherein the product is used after a bowel movement where straining can cause hemorrhoid bulges and irritation.

7. The hemorrhoid pain relief and healing pack of claim 1, wherein the fluid within the cold pack is gel or liquid.

8. The hemorrhoid pain relief and healing pack of claim 1, wherein the protective sleeve is made of ultra-soft disposable cloth; measuring approximately 3¼ inches in width, 4½ inches in length, and consisting of a 1 inch fold to allow for pocket insertion; and is infused with 10 ml of 100% natural witch-hazel.

9. The hemorrhoid pain relief and healing pack of claim 8, wherein the protective sleeve is made of other soft materials and/or is infused with an increased/decreased of the amount of witch-hazel.

10. The hemorrhoid pain relief and healing pack of claim 8, wherein the protective sleeve is stored within its own disposable packet keeping the product sanitary.

11. The hemorrhoid pain relief and healing pack of claim 8, is disposable and therefore sanitary.

12. The hemorrhoid pain relief and healing pack of claim 1, further relieves pain from swelling, itching, burning, and discomfort.

13. The hemorrhoid pain relief and healing pack of claim 1, further aides in shrinking current hemorrhoid bulge/s.

14. The hemorrhoid pain relief and healing pack of claim 1, the cold pack is frozen before use to offer cold therapy pain relief.

15. The hemorrhoid pain relief and healing pack of claim 1, the cold pack is inserted into the witch-hazel infused disposable cloth sleeve before use providing comfort and a healing astringent to further aide in healing/shrinking the hemorrhoids.

16. The hemorrhoid pain relief and healing pack of claim 1, is easy to use.

17. The hemorrhoid pain relief and healing pack comprises: a small gel filled oval cold pack;
wherein the cold pack being an oval shape of approximately 5 inches in length and 3 inches in width and made of smooth yet strong material capable of containing gel or other flexible freezable liquids under pressure; and inserted within an ultra-soft disposable cloth sleeve being approximately 3¼ inches in width, 4½ inches in length, and consisting of a 1 inch fold for pocket insertion and infused with approximately 10 ml of 100% natural witch-hazel;
the product being placed over the hemorrhoid bulge/s and conforming to their shape, provides instant cooling relief of pain due to swelling, itching, and burning, while the witch-hazel acts as a soothing astringent, aiding in the shrinking and healing of present hemorrhoids.
